(19) United States
(12) Patent Application Publication
(10) Pub. No.: US 2011/0251595 A1
(43) Pub. Date: Oct. 13, 2011

(54) ENDOVASCULAR CATHETER AND METHOD WITH HYDRAULIC BLADDER SYSTEM

(75) Inventors: Elias Habib Kassab, Grosse Pointe Shores, MI (US); Mark Edwin Zyzelewski, Kalamazoo, MI (US)

(73) Assignee: KASSAB KUGHN ENDOVASCULAR DEVICES LLC, Farmington, MI (US)

(21) Appl. No.: 12/755,864

(22) Filed: Apr. 7, 2010

(51) Int. Cl. A61M 25/00

(52) U.S. Cl. 604/528

(57) ABSTRACT

A endovascular sheath system 10 and method are provided that has an inner tube 12 which includes an outer surface 16 and an uninterrupted inner lumen 14 for introducing medical fluids or devices. A stiffening assembly 26 including a plurality of inflatable bladders 18 is disposed outside the inner tube 12 that extend axially in relation to the inner tube 12 along at least a portion of its length. At least some of the bladders 18 are inflated or deflated through a channel 20 that lies in an annular wall 24 disposed between the outer 16 and inner wall surfaces 15 of the tube 12 and when a bladder is pressurized by hydraulic pressure. When pressure is relieved, at least partial relaxation and therefore a softening of the apparatus 10 results. An actuating mechanism 32 cooperates with a proximal end 42 of the stiffening assembly 26 for controlling fluid pressure that is communicated to the plurality of bladders 18.

### BACKGROUND OF THE INVENTION

1. Field of the Invention

One aspect of the invention relates to an endovascular catheter system and method including a sheath with a stiffness characteristic imparted by a hydraulic bladder, the pressure of which is controlled extracorporeally.

2. Background Art

Endovascular surgery is a technique that is used to access many regions of the body through major blood vessels. Conventionally, a catheter is introduced through the skin into a large blood vessel such as the femoral artery. Often, the catheter carries a radio-opaque dye that can be detected by X-ray or fluoroscopic procedures. Endovascular surgery is becoming more widely used because it is minimally invasive and offers immediate advantages over more traditional, yet highly invasive surgeries.

Generally stated, a catheter is a tube that can be inserted into a body cavity, duct or vessel. Catheters typically allow drainage or the injection of a fluid or access by surgical instruments. Many uses require that the catheter be thin and flexible (a “soft” catheter or tube); in other cases it may be a larger solid tube—a “hard” catheter.

As used herein, (1) the term “guide wire” refers to a long and flexible fine spring that is used to introduce and position an intra vascular catheter; and (2) the term “sheath” refers to the outer covering of a guide wire. Unless the context suggests otherwise, “sheath” may be used interchangeably with the term “catheter”. Conventionally, the catheter or sheath is often threaded over the wire. The wire may then be withdrawn, leaving the catheter in place (the Seldinger Technique)—a procedure that is used to obtain access to blood vessels and other hollow organs. Seldinger S., Catheter Replacement of the Needle in Percutaneous Arteriography; a New Technique, Acta Radiologica (5); 368-76 (1953).

During interventional vascular procedures, situations often arise where a catheter needs to be advanced through tortuous paths, in which it may be difficult to steer a guide wire or other interventional device along the interstices of a winding vessel. For example, one area in the vascular geometry that causes issues related to advancing a device is at the interface of the femoral and aortic vessels. During peripheral procedures, the entry location for devices is often the femoral artery on the side of the body opposite the area of concern. The device must then pass over the femoral arch and into the opposite femoral artery. When an interventional device used during the procedure advances towards the femoral/aortic interface, for example, buckling of the device often occurs. Conventional interventional devices tend to buckle under the axial force reaction of the vessel wall to the pressures of insertion. Resistance to passage and consequent bending of the device often occurs proximal to the tortuous anatomy.

There are several reasons why this situation is difficult to overcome with many conventional techniques:

1. The geometry of a guide wire. A guide wire must have some flexibility to be able to steer through the vascular anatomy without injuring a vessel. A guide wire is typically a small diameter wire (e.g., 0.014″) and is pliable and soft. When the guide wire is advanced through any tortuous anatomy, due to the small size of the wire, it has little ability to resist buckling. The proximal section of the wire typically buckles, and very little if any force is transmitted to the distal end of the wire.

2. The flexibility of the guiding catheter or sheath. Guiding catheters and sheaths are conventionally made from reinforced extruded plastic tubing. These devices are soft and generally provide little resistance to buckling.

3. The intervention devices that are used during the procedure. The intervention devices (e.g. stent delivery systems and bladders) are pliable along their axial shaft, and easily buckle when an axial force is applied.

Thus, most traditional devices and techniques do not provide a solution to the buckling that occurs when a tortuous anatomy is encountered during an endovascular procedure. If a device cannot be advanced through this tortuous anatomy, the procedure may not be able to be performed, and the outcome for the patient may be compromised.

It is known that when the leading edge of a sheath encounters a tight lesion, there is a loss of kinetic energy. Some approaches solve this problem by using a hydrophilic material that is applied to the sheath. The problem with this approach, however, is that the surgeon's hands tend to slip over the outside surface of the sheath.

As used herein, the term “distal” designates a direction away from the operator toward an end of the catheter closest to the anatomical site of interest. Conversely, the term “proximal” refers to a direction towards an operator and the location where a catheter system may be injected into a patient.

Among the U.S. and foreign patent documents that were considered before filing this application are: U.S. Pat. Nos. 5,599,326; 7,226,466; 7,273,487; 2006/0235502; 2006/0258987; 2006/0264907; 2007/0049899; 2007/0060880; 2008/0045895; and 2008/0172037.

### SUMMARY OF THE INVENTION

Against this background it would be desirable to have a sheath with a simple design that is easy to use in patients who require an interventional vascular procedure. Preferably, such a sheath would be intuitive to use and require minimal surgical finesse, yet be able to negotiate a tortuous path that may include calcified lesions and fibrosed areas, swiftly and with repeatability if desired, without disrupting adjacent tissue.

To meet these and related needs, an endovascular sheath apparatus is provided that comprises an inner tube which includes a clear, uninterrupted lumen for introducing medical fluids or devices and an outer surface. Disposed outside the inner tube are one or more inflatable bladders that extend axially in relation to the inner tube along at least a portion of its length.

The one or more bladders are inflated or deflated through a channel(s) that lies in an annular wall disposed between the outer and inner wall surfaces of the tube. When the bladder system is pressurized, preferably by hydraulic pressure, a stiffening characteristic is thereby provided to the tube. When the pressure is released, at least partial relaxation and therefore a softening of the tube results.

One aspect of this disclosure includes a device and method that reduces or prevents unwanted buckling of interventional devices. An embodiment of the subject device allows the transmission of axial forces towards the distal end of the catheter assembly without kinking or buckling.

Preferably, the disclosed sheath device reduces the risk of vascular perforation by offering a tip design that does not ablate tissue ahead of its distal end, thereby avoiding inadvertent rupture of tissue that may be contacted. In some circumstances, it may be desirable to have a differential level of stiffness along the length of a sheath. For example, the distal region of the sheath might desirably be fluffy or soft. This would reduce the risk of trauma that might otherwise be caused by interference between the distal end of the sheath and a tight turn in an arterial wall, for example.

Ideally, the surgeon would like to be able to determine without interchanging sheaths whether a particular sheath would be stiff or not stiff (compliant) in an intermediate region or in a region proximate its distal end. Thus, one aspect of the disclosure includes a sheath that has a changing modulus of elasticity along its length. In some embodiments, the sheath may have a modulus of elasticity or flexural modulus along its length that could be discretely or gradually changed.

Another facet of the disclosure is the provision of a sheath that has the capability to change its outside diameter along its length so that, for example, the outside diameter may be less at the distal than at the proximal end.

Cooperating with the stiffness component is an actuating mechanism, such as a valve system and a pressure regulator that are provided extracorporeally.

### BRIEF DESCRIPTION OF THE DRAWINGS

FIG. 1 is a side elevation or top plan view of an embodiment of an endovascular catheter system with a stiffening assembly including a hydraulic bladder;

FIG. 1A is an enlargement of a depicted portion of the system illustrated in FIG. 1;

FIGS. 2A-E are transverse sectional views across the system at positions A-E that are indicated in FIG. 1;

FIG. 3 is a quartering perspective view of a hub and extensions therefrom that are provided at a proximal end of an embodiment of the disclosed system;

FIG. 4 is a longitudinal cross-sectional view of the embodiment depicted in FIG. 3 along the line 4-4 thereof;

FIG. 5 enlarges a portion of FIG. 4;

FIG. 6 is a longitudinal sectional view of a portion of the proximal end regions of an embodiment of a stiffening assembly, including a bladder system;

FIG. 7 resembles the view of FIG. 6 but depicts additional structural detail of the bladder system;

FIG. 8 illustrates a transverse sectional and perspective view of an embodiment of the disclosed a stiffening assembly, illustrating the bladder system in an inflated state;

FIGS. 9A-D are transverse sectional views of alternate embodiments of the bladder system.

### DETAILED DESCRIPTION OF THE PREFERRED EMBODIMENT(S)

This disclosure provides a method and system that reduces or prevents catheter buckling by allowing the transmission of axial forces towards its distal end.

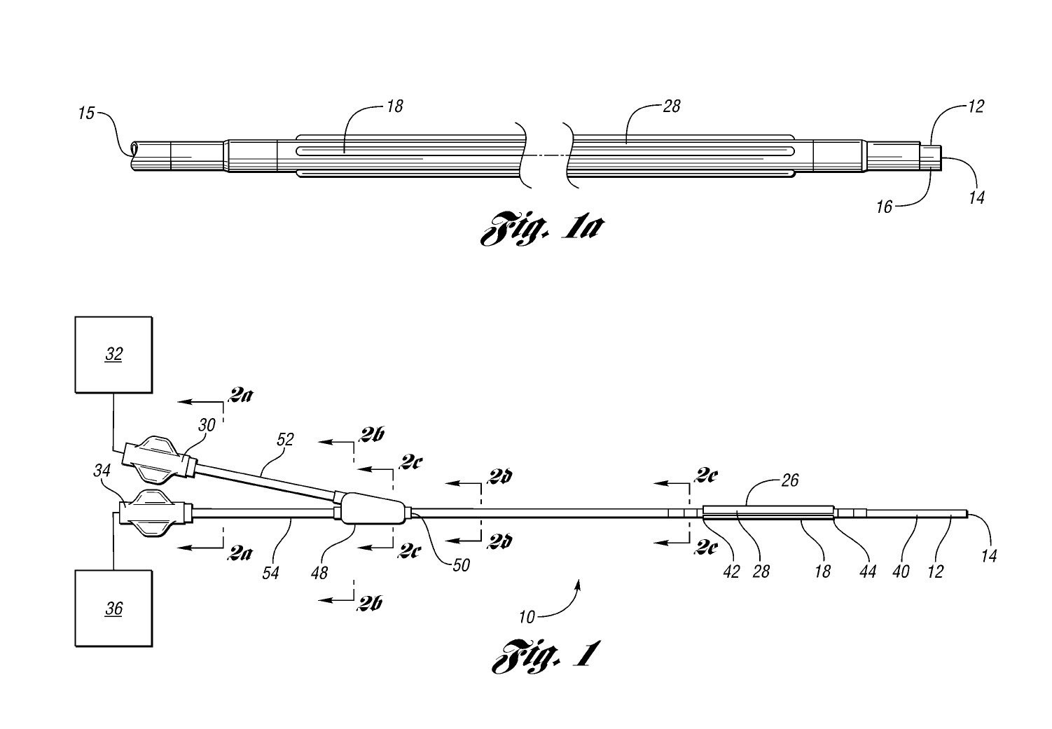

In one embodiment (FIGS. 1, 1(A)), an endovascular catheter system 10 is provided that comprises an inner tube 12 with a lumen 14 defined within an inner wall surface 15. The tube 12 has an outer surface 16. Medical fluids or devices can be introduced or removed along the lumen 14. Disposed outside part of the inner tube 12 is a stiffening assembly 26 with one or more (a number (N)) inflatable bladders 18 (where 1<N<20) that extend axially in relation to the inner tube 12 along at least a portion of its overall length (FIG. 1).

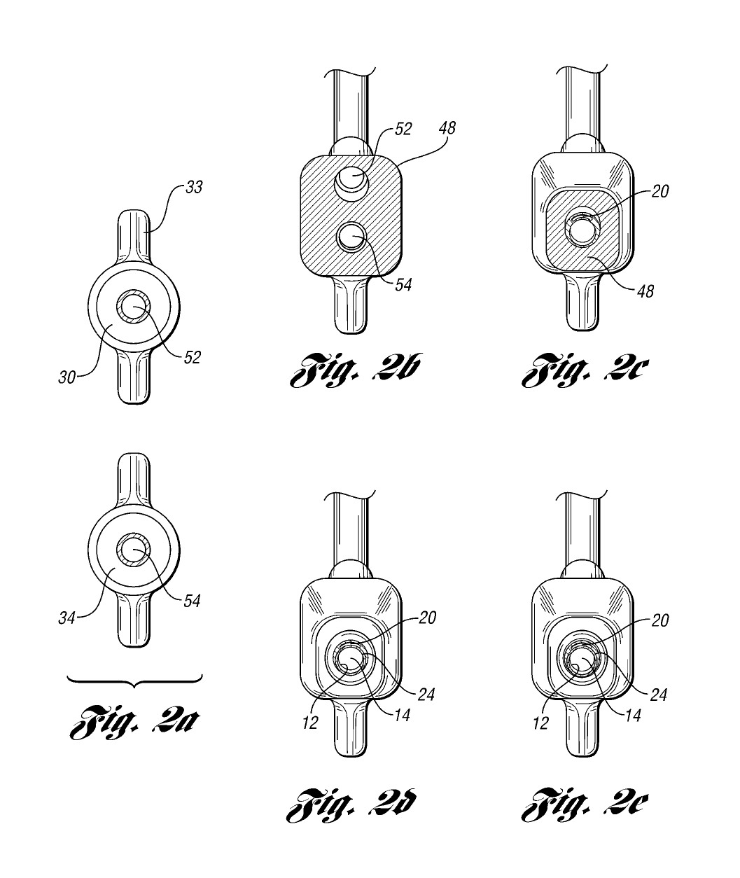

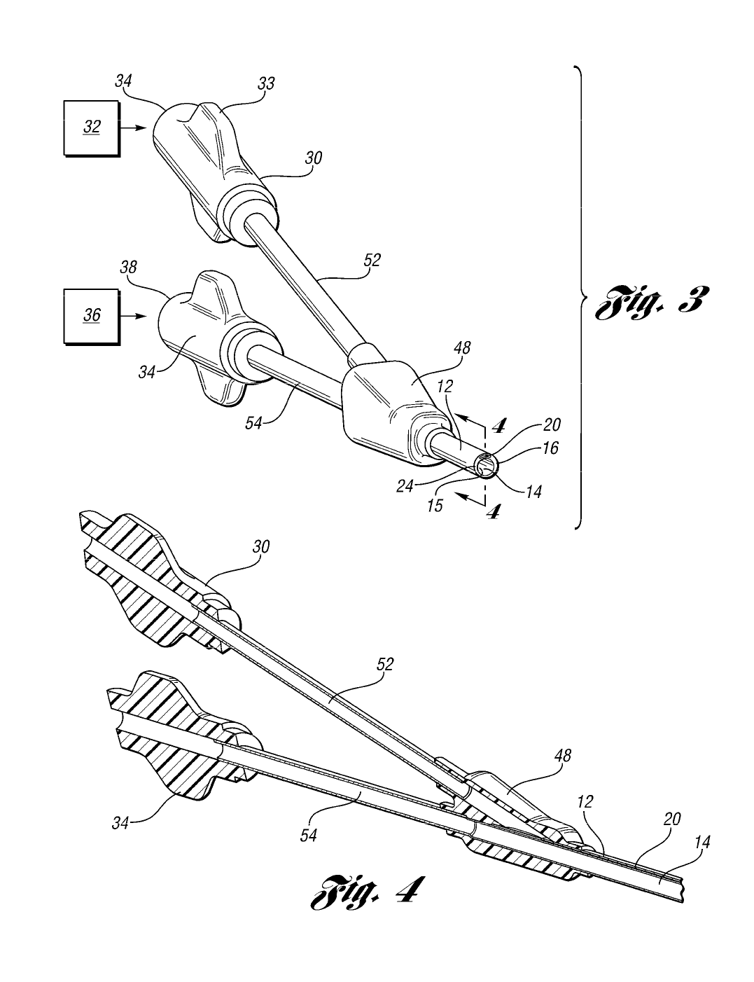

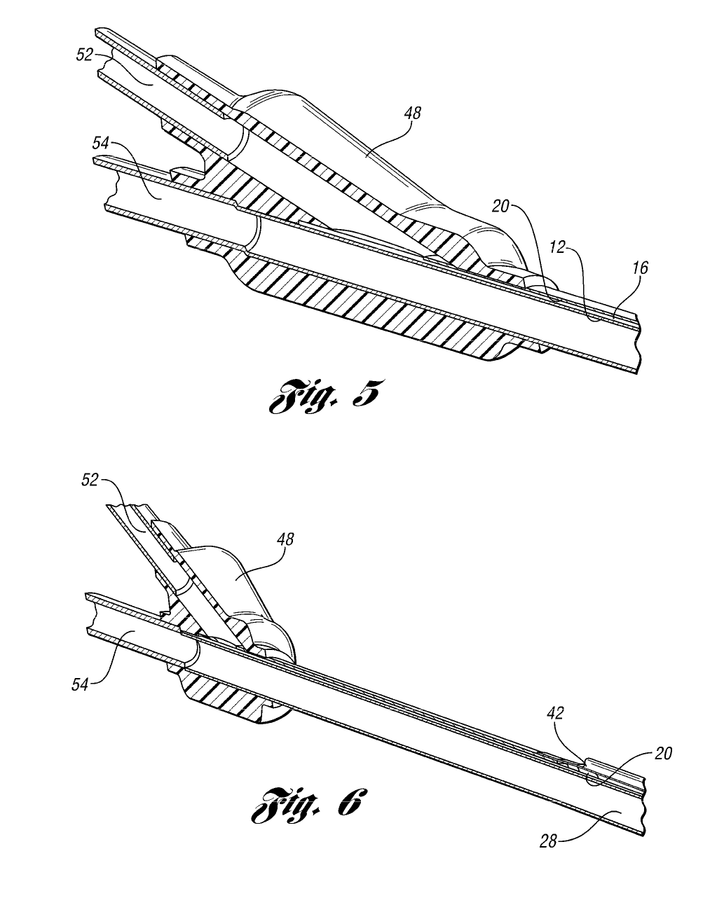

The bladders 18 are inflated or deflated through a number (M) of channels 20 (where 1<M<8) that are associated with or lie in an annular wall 24 disposed between the outer 16 and inner wall surfaces 15 of the tube 12 (FIGS. 2-7). Fluid (such as a saline solution) from the channel 20 may be ducted to the bladders 18 through apertures defined in the annular wall 24 of the tube 12. When the bladder 18 is pressurized by hydraulic pressure, a stiffening characteristic is thereby imparted to the tube 12. When the pressure is released, at least partial relaxation and therefore a softening of the tube 12 results. This helps prevent the mid section 28 of the tube 12 from being kinked, and imbues stiffness to the sheath apparatus 10. Softening facilitates removal of the catheter system from a tortuous vasculature.

Cooperating with the stiffness assembly 26 is an extracorporeal pressure hub section 30 (FIGS. 1, 3-4) that interfaces with a pressure regulator, controller and source of pressurized fluid 32. In one embodiment, the pressure hub section 30 may include a manifold having an inlet that communicates with the pressure regulator and multiple outlets, at least some of which may communicate with some or all of the number (M) of channels 20 that lie in the annular wall 24 of the inner tube 12.

An inner lumen proximal hub section 34 communicates with the inner lumen 14. In one embodiment, the inner lumen hub 34 is monolithic—a one piece structure that houses a one way valve 36 at or near its proximal end 38 (FIG. 3). This valve 32 prevents the flow of blood out of the inner tube 12, but allows the introduction of interventional devices (guide catheters, wires, etc.) into the vasculature via the inner tube 12. Optionally, hub 34 may include other sub components with one or more removable valves or other connectors, such as Tuohy Borst adapters. The proximal end 38 of hub 34 may also connect with a luer lock system for flushing or injecting fluids such as contrast fluids into the catheter system 10.

In one aspect, the subject apparatus and method provide the interventionalist with a catheter system 10 that has modifiable stiffness at various regions along bladder length when desired to facilitate a medical procedure. This facility is enabled by bladders 18 that are provided in those regions at which stiffness is desired.

The disclosure includes variations in diameter, length, wall thickness and materials of the inner tube 12 and stiffening assembly 26 which influence the resistance offered by the catheter system 10 to deformation (extension, compression, twisting or bending) of the inner tubular section 12, such that the catheter system can be made to be more or less pushable and/or resistant to kinking. If desired, the inner tube 12 may include a polymer that is reinforced by one or more braided layers. This stiffening, including customizing or tuning the characteristics of flexibility, suppleness and pliability, will allow the interventionalist to more readily, reproducibly and predictably transmit axial force to the distal end region 40 of the catheter system 10 and facilitate the passage of interventional devices such as stents and balloons and pharmaceuticals through a tortuous anatomy. If desired, such tuning may be done at different times during an endovascular procedure.

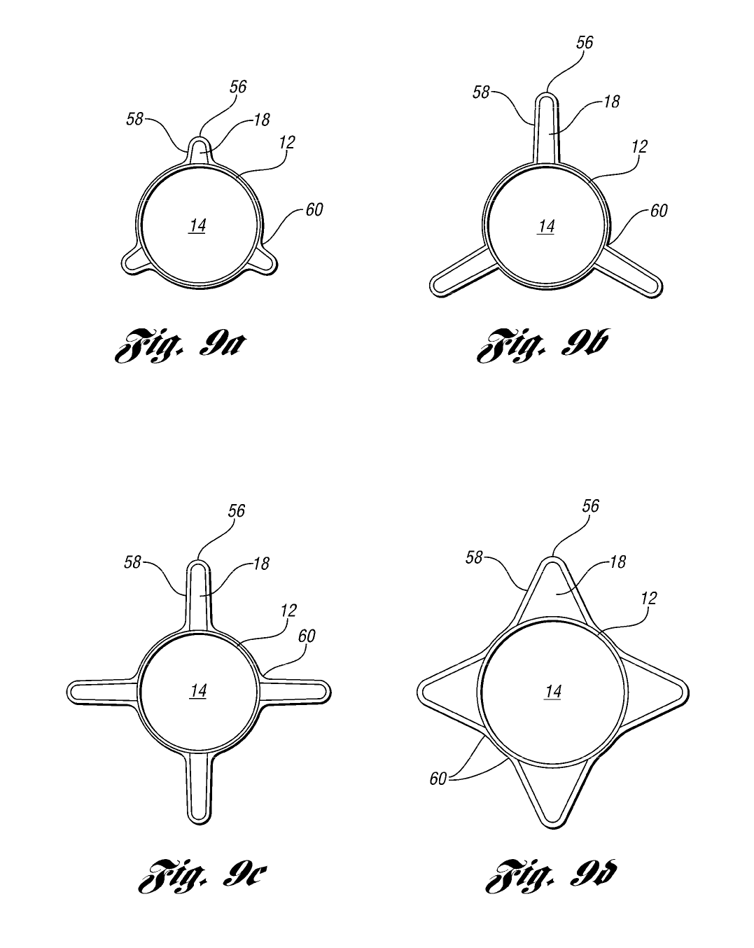

In one aspect, the disclosed catheter system includes a sheath 12 having an I.D. of 5-9 FR (7 FR preferred) with coaxial, multiple lumens (e.g., dual lumens 14, 20) (FIGS. 2, 4-8). One (inner) lumen 14 allows passage of various devices and/or fluids, including pharmaceuticals and other solutions. Another lumen or channel 20 is defined for example in the wall 24 of the inner tube 12. That lumen 20 permits inflation and deflation of a bladder or bladders 18 by hydraulic pressure applied in the bladders 18 of the stiffening assembly 26.

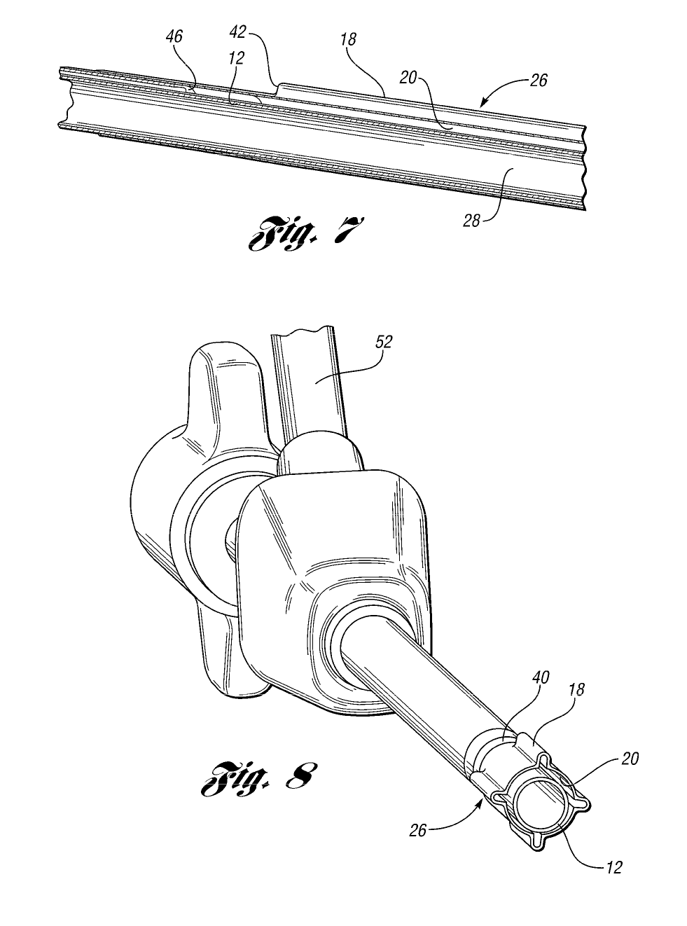

In an exemplary embodiment, the dual lumen sheath 10 is selectively stiffened by a bladder system 18 that is affixed to the outer surface or embedded in the wall 24 of tube 12. One or more side lumens duct the inflating fluid 22 from the source of pressure 32 to the proximal 42 and distal 44 ends of the bladder or balloon subassembly 18 (FIG. 1). Inflation, pressure (and therefore stiffness) maintenance and deflation occur through a port 46 (FIG. 7) disposed in a side or end wall of a channel 20 by communicating the fluid (hydraulic—preferably—or pneumatic or combined pneumatic and hydraulic) therethrough. If desired, a given bladder 18 could be inflated or deflated by hydraulic fluid that enters the bladder through multiple ports 46 extending at least partially along its length into the bladder from a feeder channel 20 that reposes in the annular wall 24. In such an assembly, the cross-sectional structure of the sheath 10 effectively provides an axial or longitudinal stiffening assembly or rib system 26 that resists bending when an inflation pressure is delivered to the fluid.

Within the scope of this disclosure, multiple alternative embodiments of a stiffening subassembly 26 are contemplated (see for example, FIG. 9). In all embodiments, the sheath 10 has a dual lumen cross-section in its central shaft area—one larger center lumen 14 for device or medicinal/pharmaceutical fluid media insertion and removal, and one or more smaller side lumens or channels 20 for inflation, pressure maintenance or deflation of the stiffening assembly or bladders (see also FIGS. 2-8).

In an illustrative embodiment, the proximal hub 48 (FIGS. 1-5, 8) is attached to the proximal end 50 of the inner tube 12. The proximal hub 48 optionally has at least two inlet ports 52, 54. One is the device medicinal/fluid port 54, which creates a pathway for devices or pharmaceutical fluids to enter the center lumen 14 and advance into or escape from the vasculature. The hydraulic port 52 communicates hydraulic fluid (e.g., saline solution) to the stiffening assembly 26 for bladder inflation/pressure maintenance/deflation.

The proximal hub 48 (FIGS. 2-3) mates with each port 52, 54 through sealed connections that allow simultaneous device insertion and bladder stiffening by inflation/deflation. The proximal hub 48 forms a sealed connection with the bladder 18, and a sealed connection to the center lumen 14.

As noted earlier, the one or more side lumens or channels 22 in alternative embodiments interface with one or more stiffening bladders 18. The side lumen(s) 22 extend at least partially from the sealed connection at the proximal hub 48 towards the proximal end 42 of the bladders 18 in the stiffening assembly 26. The proximal end 42 of the stiffening bladder system 18 is located adjacent to or near the distal end of the side channel 20. This allows a fluid medium to be introduced through the inflation/deflation port 52, pressurize the inside of the one or more stiffening bladders 18 and thus expand the bladders to a distended configuration.

When pressure is applied to the stiffening assembly 26, the bladders 18 expand. The expanded cross-section of the bladders around the tube 12 acts as one or more stiffening ribs along some or all of the length of the inner tube 12. This in turn increases the cross-sectional stiffness of the sheath 10, preventing the sheath 10 from buckling when in use with interventional devices.

The length and geometry of the stiffening bladder 26 may be presented in many varieties and combinations. FIG. 9A illustrates a pre-inflation condition, while FIG. 9B illustrates a condition in which a bladder system 18 (3 ribs) is stiffened by pressurization. Similarly, for FIGS. 9C-D (4 ribs).

It will be appreciated that the multiple bladders 18 extend axially and radially from the inner tube 12. Preferably, the bladders 18 are equally spaced radially about the outer perimeter 16 of the inner tube 12.

Each bladder 18 includes a rounded head portion 56, a neck portion 58 that extends therefrom towards the inner tube 12 and a shoulder portion 60 that transitions between the neck portion 58 and an outside surface of the inner tube 12. It will be appreciated that in FIG. 9D the shoulder portions 60 of adjacent bladders 18 merge, or almost merge. In one alternate embodiment, the bladders may coalesce to form an annular bladder that surrounds the inner tube 12.

In FIG. 9B, the neck portion 58 of bladder 18 extends radially outwardly under pressure exerted by the fluid in the fluid channel 20. Accordingly, the distended neck portions 58 may serve as spacer members in that after radially outwardly directed pressure is exerted against the inner wall of a narrow artery, the inner tube 14 is thereby displaced to and is held approximately axially and centrally within the artery.

If desired, a film of a material may be provided on the outside surface(s) of the bladders 18 and/or inner tube 12 that minimizes friction and otherwise renders glissile the passage of the catheter system 10 through the vasculature.

Saline solution and/or a contrast fluid may be used to pressurize the balloons 18.

In practice, the stiffening assembly 26 may be at least partially evacuated before insertion into the patient. Following this step, the catheter system 10 may be rendered more or less rigid once in place by regulating fluid pressure. Preferably, the range of fluid pressures lies between 0-10 atmospheres which in all cases is less than the burst pressure of the bladders 18. Conversely, before device removal, fluid pressure is reduced by at least partially purging the bladder system 26 of the stiffening fluid. A negative pressure (vacuum) evacuation system may be provided in combination with the pressure regulation system 32 in order to drain the fluid from the stiffening assembly 26. It is contemplated that within the scope of this disclosure, some bladders 18 in a given assembly may be provided in axial configuration such that the pressure in various bladders 18 may differ. This may provide the opportunity to pre-shape the sheath assembly 10 such that it bends more readily in one plane than in another. In such approaches, pressure delivery to individual bladders 18 may be isolated from pressures delivered to a neighboring bladders 18.

It will be appreciated that the bladders 18 may be provided axially on or near the outer wall 16 of the inner tube 12. Optionally, the channel 18 that pressurizes the stiffening system 26 may cross the annular wall 24 of the inner tube 12 and lie within the inner wall surface 15 of the inner tube 12. Optionally, the longitudinal axis of the one or more bladders 18 may be oriented helically so that they wrap partially or completely around the inner tube 12.

Thus, some bladders 18 may be mounted in recesses within a wall 24 of the inner tube 12 or may be provided outside the inner tube 12 in helical, bent, or discontinuing configurations. Locations along the length of the catheter 10 can be preselected and predetermined to engender a preferred curvature or preferential bending at desired regions along the catheter 10. Such properties may enable the sheath 10 to be preferentially stiffened after it is placed in situ.

Numerous materials can be used to make the catheter system 10, including several composites that employ construction techniques with which those in the art are conversant.

Preferably, markers that are radio-opaque are incorporated in proximal 42 and distal ends 40 of the catheter system 10 or stiffening assembly 26 that allow for positioning and perception of the ends of the balloons 18.

Although the cross section of the tube 12 and inner lumen 14 is depicted as circular, it will be appreciated that the invention is not so limiting. For example, if it is desired that the bladder-tube combination 18, 12 be rounded, then troughs or furrows may be defined in the outside surface 16 of the inner tube 12 to accommodate the bladders 18. Such accommodation may or may not translate into an inner tube cross section with an outer wall 16 that may include lands with valleys or troughs therebetween for accommodating the bladders 18. In some cases, circumferentially disposed bladders 18 are radially separated by 90, 120 degrees or other amounts of radial displacement.

Here is a list of reference numerals used in this disclosure and the components to which they relate:

| No. | Component |
|---|---|
| 10 | Catheter System |
| 12 | Inner Tube |
| 14 | Center Lumen |
| 15 | Inner Wall Surface |
| 16 | Outer Wall Surface |
| 18 | Inflatable Bladders |
| 20 | Channel |
| 22 | Side Lumen |
| 24 | Annular Wall |
| 26 | Stiffening Assembly |
| 28 | Mid Section |
| 30 | Pressure Hub |
| 32 | Pressure Controlling/Actuating System |
| 33 | Handle |
| 34 | Proximal Hub |
| 36 | One Way Valve (Inner Lumen) |
| 38 | Proximal End of 34 |
| 40 | Distal End of 10 |
| 42 | Proximal End of 20 |
| 44 | Distal End of 20 |
| 46 | Port in 18 |
| 48 | Proximal Hub |
| 50 | Proximal End of 12 |
| 52 | Hydraulic Port |
| 54 | Device/Fluid Port |
| 56 | Rounded Head |
| 58 | Neck |
| 60 | Shoulder |

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

1. An endovascular sheath apparatus comprising:
an inner tube which includes an uninterrupted inner lumen for introducing medical fluids or devices, stents, wires, balloons or other atherectomy devices; and
a stiffening assembly including one or more inflatable bladders disposed outside the inner lumen that extend axially in relation to the inner tube along at least a portion of its length, so that at least some of the bladders are inflated or deflated through one or more channels that lie in or outside an annular wall disposed between the outer and inner wall surfaces of the inner tube and when the one or more bladders are pressurized, a stiffening characteristic is provided to the inner tube so that when pressure is alleviated, at least partial relaxation and therefore a softening of the sheath results.

2. The sheath apparatus of claim 1, further including:
an inner tube hub that is attached to a proximal end of the inner tube; and
a valve system detachably connected to the inner tube hub, the valve system including a one-way valve by which the medical fluids may be ducted into the inner tube, the valve preventing the flow of fluids out of the endovascular sheath, but allowing the introduction of interventional devices and fluids into the vasculature.

3. The sheath apparatus of claim 1, wherein the stiffening assembly comprises a number (N) of bladders, where 1<N<20.

4. The sheath apparatus of claim 1, further comprising:
a hydraulic port and a device/fluid port at a proximal end of the sheath apparatus through which hydraulic fluid is delivered.

5. The sheath apparatus of claim 4, wherein the hydraulic port communicates with a channel that is associated with each inflatable bladder.

6. The sheath apparatus of claim 1, wherein the stiffening assembly comprises a number (M) of channels along which fluid may flow, where 1<M<8.

7. The sheath apparatus of claim 6, wherein the fluid includes saline solution.

8. The sheath apparatus of claim 7, wherein the fluid channels are defined outside an outer wall surface of the inner tube.

9. The sheath apparatus of claim 1, further comprising an actuating mechanism that cooperates with a proximal end of the stiffening assembly for controlling fluid pressure that is communicated to the one or more channels and bladders in the stiffening assembly for creating at least partially around the inner lumen a stiffness characteristic that is influenced by the actuating mechanism, the actuating mechanism communicating with a pressure regulating device that controls fluid pressure within a fluid channel.

10. The sheath apparatus of claim 1, wherein the stiffening assembly comprises multiple bladders that extend axially and radially from the inner tube and that are equi-spaced thereabout.

11. The sheath apparatus of claim 10, wherein fluid pressure within the multiple bladders is unequal, thereby imparting a preferred curvature to the sheath.

12. The sheath apparatus of claim 11, wherein each bladder includes a rounded head portion, a neck portion that extends therefrom towards the inner tube, and a shoulder portion that transitions between the neck portion and an outside surface of the inner tube.

13. The sheath apparatus of claim 12, wherein the shoulder portions of adjacent bladders merge.

14. The sheath apparatus of claim 12, wherein the neck portion extends radially outwardly under pressure exerted by fluid in the fluid channel.

15. The sheath apparatus of claim 12, wherein the neck portion extends circumferentially under pressure exerted by fluid in the fluid channel.

16. The sheath apparatus of claim 14, wherein the sheath apparatus has an external diameter that increases in response to forces exerted radially outwardly by fluid in the fluid channels and decreases when fluid pressure is relieved.

17. A method of guiding a sheath through a vascular passageway, the method comprising the steps of:
(A) inserting a distal end of the sheath into a vascular structure;
(B) providing an inner tube which includes an outer surface and an uninterrupted inner lumen for introducing medical fluids or devices; and
a stiffening assembly including a plurality of inflatable bladders disposed outside the inner lumen that extend axially in relation to the inner tube along at least a portion of its length, so that at least some of the bladders can be inflated or deflated through a channel and when a bladder is pressurized, a stiffening characteristic is thereby provided to the tube and when the pressure is released, at least partial relaxation and therefore a softening of the apparatus results;
(C) adjusting the stiffness of at least a portion of the sheath by applying a fluid pressure to the plurality of inflatable bladders to selectively stiffen at least a portion of the inner tube.

18. The method of claim 17, wherein the adjusting step comprises stiffening at least one of the inflatable bladders associated with the inner tube when additional column strength is needed to overcome an obstruction or constriction in the vascular passageway.

19. The method of claim 17, wherein the adjusting step comprises reducing the stiffness of the inner tube by relieving fluid pressure in the stiffening assembly when the sheath is being pushed through a tortuous part of the vascular system.

20. The method of claim 17, wherein the amount of fluid pressure and stiffness are varied while the sheath is being pushed through the vascular passageway.

21. A method of providing intravascular delivery of a sheath apparatus, comprising steps of:
(A) providing an inner tube that includes a lumen for introducing medical fluids or devices and an outer surface;
(B) at least partially surrounding the inner tube with a stiffening assembly including one or more axially extending bladders;
(C) communicating a source of fluid pressure with the stiffening assembly, the bladders of the stiffening assembly extending radially outwardly from the inner tube in response to fluid pressure and thus imparting to the stiffening assembly and the inner tube a stiffness characteristic; and
(D) regulating fluid pressure imparted to the bladders in order to influence a stiffness characteristic of the inner tube without incursion of its inner wall into the lumen of the inner tube.

22. The sheath apparatus of claim 3 wherein at least some of the one or more inflatable bladders have a longitudinal axis that is oriented helically so that the one or more bladders wrap partially or completely around the inner tube.
